(19) United States
(12) Patent Application Publication
(10) Pub. No.: US 2023/0096607 A1
(43) Pub. Date: Mar. 30, 2023

(54) Systems and Methods for Calculating Carbon Emission Reductions

(71) Applicant: Mesa Natural Gas Solutions, LLC, Loveland, CO (US)

(72) Inventors: Marcin Pawel Pankau, Loveland, CO (US); Zachary Alan Soukup, Oklahoma City, OK (US)

(21) Appl. No.: 17/485,863

(22) Filed: Sep. 27, 2021

(51) Int. Cl. F23G 7/08; F23N 5/00; G01N 33/00

(52) U.S. Cl. CPC F23G 7/085; F23N 5/003; G01N 33/004; F23N 2241/12

(57) ABSTRACT

A process for calculating a carbon emission reduction comprises calculating a carbon dioxide equivalent (CO2e) output associated with using a measured quantity of wellhead gas in generators to produce electricity, determining a carbon dioxide equivalent (CO2e) emission associated with flaring the measured quantity of wellhead gas, and calculating a carbon emission reduction as the difference between the determined CO2e emission and the calculated CO2e output.

### TECHNICAL FIELD

The present disclosure is directed to systems and methods for calculating a carbon emission reduction through converting flared gas to electricity.

### BACKGROUND

When valuable hydrocarbons are produced from a subsurface well, byproduct wellhead gas may also be produced. If the wellhead gas is deemed uneconomical to collect and sell, or would otherwise present a safety problem, operators will often flare the gas.

### SUMMARY

The present disclosure relates to systems and methods for calculating a carbon emission reduction by using wellhead gas as a fuel supply to one or more generators to produce electricity instead of flaring the wellhead gas to the atmosphere.

In accordance with one aspect of the present disclosure, a process is provided for calculating a carbon emission reduction by calculating a carbon dioxide equivalent (CO2e) output associated with a quantity of wellhead gas used as a fuel supply to a generator that produces electricity, determining a carbon dioxide equivalent (CO2e) emission associated with flaring the same quantity of wellhead gas via a flare stack system, and calculating the carbon emission reduction based on a comparison between the calculated CO2e output and the determined CO2e emission. In some implementations, calculating the carbon emission reduction comprises calculating the difference between the determined CO2e emission and the calculated CO2e output.

The process may further include measuring, in real time, the quantity of wellhead gas used as the fuel supply to the generator that produces electricity and/or using the measured quantity of wellhead gas used as the fuel supply to the generator as the same quantity of wellhead gas flared via the flare stack system. In some implementations, determining the CO2e emission includes modeling the CO2e emission using manufacturer and/or operational data associated with the flare stack system. In some implementations, calculating the CO2e output further includes calculating a first Carbon Mass based at least partially on the quantity of wellhead gas and an analysis of the wellhead gas. In some implementations, calculating the CO2e output further includes determining a Known Carbon Monoxide (CO) and Volatile Organic Compounds (VOC) Emission based at least in part on the first Carbon Mass and operational data for the generator. In some implementations, calculating the CO2e output further includes calculating a carbon dioxide (CO2) output based at least in part on the first Carbon Mass and the Known CO and VOC Emission. In some implementations, calculating the CO2e output further includes determining a Known Methane Emission based at least in part on the quantity of methane in the VOC. In some implementations, calculating the CO2e output is based at least in part on the calculated CO2 output and the Known Methane Emission.

In some implementations, determining the CO2e emission further includes determining a second Carbon Mass based at least partially on the same quantity of wellhead gas and an analysis of the wellhead gas. In some implementations, determining the CO2e emission further includes determining a Modeled carbon monoxide (CO), carbon dioxide (CO2), nitrogen oxide (NO) and Volatile Organic Compounds (VOC) Emission based at least in part on the second Carbon Mass and manufacturer and/or operational data for the flare stack system. In some implementations, determining the CO2e emission further includes determining a Modeled (CO2) Emission based at least in part on the Modeled CO, CO2, NO and VOC Emission.

In accordance with another aspect of the present disclosure, a process is provided for calculating a carbon emission reduction by calculating a carbon dioxide equivalent (CO2e) output associated with using a measured quantity of wellhead gas in generators to produce electricity, determining a carbon dioxide equivalent (CO2e) emission associated with flaring via a flare stack system a quantity of wellhead gas equal to the measured quantity of wellhead gas, and calculating the carbon emission reduction as the difference between the determined CO2e emission and the calculated CO2e output. In some implementations, determining the CO2e emission includes using operational data associated with flaring via the flare stack system the quantity of wellhead gas. In some implementations, the process further includes periodically calculating the carbon emission reduction on a given time interval, summing up the carbon emission reduction periodically calculated on the given time interval over a designated period of time to determine a carbon dioxide equivalent reduction over the designated period of time.

### BRIEF DESCRIPTION OF THE DRAWINGS

For a more complete understanding of the systems and methods of the present disclosure, reference is now made to the following description, taken in conjunction with the accompanying drawings, in which:

FIG. 1 depicts a schematic view of a process for calculating a carbon emission reduction according to the present disclosure by comparing calculated carbon dioxide equivalent output from a generator system to determined carbon dioxide equivalent emissions from a flare stack system.

FIG. 2A depicts a block diagram of an implementation of an emission tracking platform according to the present disclosure.

FIG. 2B depicts a block diagram of another implementation of an emission tracking platform according to the present disclosure.

FIG. 2C depicts a block diagram of yet another implementation of an emission tracking platform according to the present disclosure.

### DETAILED DESCRIPTION

Flaring wellhead gas emits air pollutants, including methane and carbon dioxide, which are known greenhouse gases. Flaring is also an inefficient combustion process, resulting in the emission of unburnt wellhead gas, including methane. Generators that utilize wellhead gas as a fuel source may be used to generate electricity rather than the traditional method of flaring the wellhead gas.

The various implementations of the devices, systems and methods described herein may use a combination of hardware and software. These implementations may use computer programs executing on programmable computing devices, and each programmable device may include at least one processor, an operating system, one or more data stores (including volatile memory or non-volatile memory or other data storage elements or a combination thereof), at least one communication interface and any other associated hardware and software that is necessary to implement the functionality described herein. As non-limiting examples, the computing device may be a server, a network appliance, an embedded device, a computer expansion module, a personal computer, a laptop, a personal data assistant, a cellular telephone, a smart-phone device, a tablet computer, a wireless device or any other computing device capable of being configured to implement the methods described herein. The particular implementation depends on the application of the computing device.

In some implementations, the communication interface may be a network communication interface, a USB connection or another suitable connection. In other implementations, the communication interface may be a software communication interface, such as those for inter-process communication (IPC). In still other implementations, there may be a combination of communication interfaces implemented as hardware, software, and a combination thereof.

In at least some of the implementations described herein, program code may be applied to input data to perform at least some of the functions described herein and to generate output information. The output information may be applied to one or more output devices, such as for display or for further processing.

In some implementations described herein, computer programs may be implemented in a high-level procedural or object-oriented programming and/or scripting language or both. Accordingly, the program code may be written in C, Java, SQL or any other suitable programming language and may comprise modules or classes, as is known to those skilled in object-oriented programming. However, other programs may be implemented in assembly, machine language or firmware as needed. In either case, the language may be a compiled or interpreted language. The computer programs may be stored on a storage media (e.g., a computer readable medium such as, but not limited to, ROM, magnetic disk, optical disc) or a device that is readable by a general or special purpose computing device.

The program code, when read by the computing device, may configure the computing device to operate in a new, specific and predefined manner in order to perform at least one of the methods described herein.

Furthermore, some of the programs associated with the system, processes and methods described herein are capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. In alternative implementations, the medium may be transitory in nature such as, but not limited to, wire-line transmissions, satellite transmissions, internet transmissions (e.g., downloads), media, digital and analog signals, and the like. The computer usable instructions may also be in various formats, including compiled and non-compiled code.

The systems and methods of the present disclosure determine, in real time, how much carbon dioxide equivalent (CO2e) a well site operator reduces or removes from the atmosphere by using generators to produce electricity instead of flaring the wellhead gas. That reduction can be used to calculate a carbon emission reduction, which may or may not have an assigned monetary value.

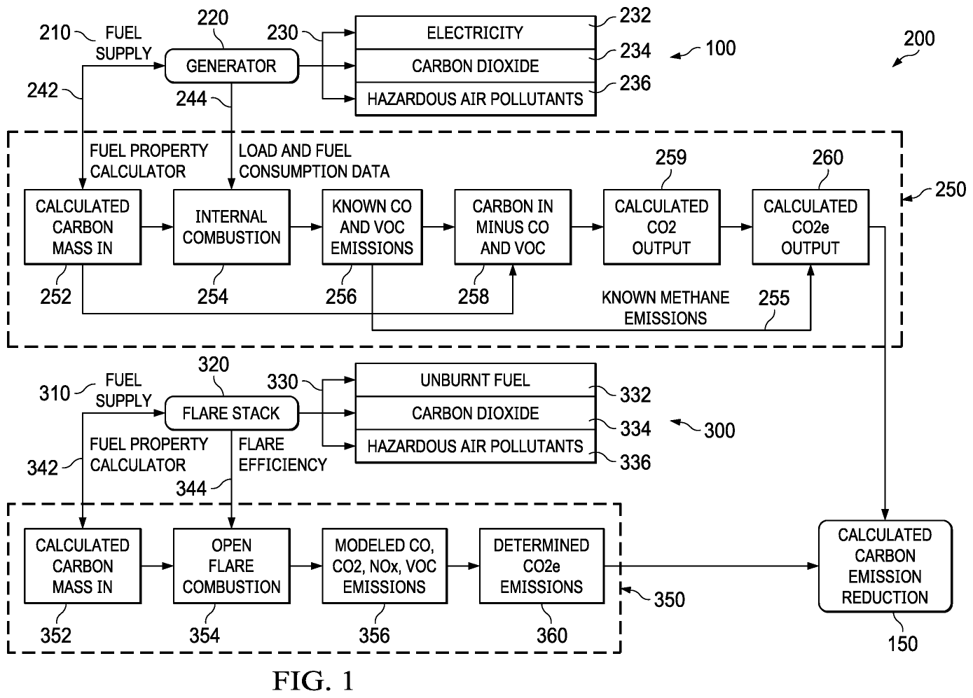

With reference now to FIG. 1, in general, the process 100 for calculating a carbon emission reduction 150, in real time, compares the calculated carbon dioxide equivalent output 260 from a generator system 200 to the determined carbon dioxide equivalent emissions 360 from a flare stack system 300. The calculated carbon emission reduction 150 corresponds to the amount of carbon dioxide equivalent reduction.

Referring first to the generator system 200, a measured wellhead fuel supply 210 flows to the generator 220. In various implementations, the wellhead fuel supply 210 may be measured by a volumetric flowmeter, a gas meter, a gas metering skid, or any other device operable to measure the wellhead fuel supply 210. As the generator 220 uses the wellhead fuel supply 210, it produces several outputs 230, including electricity 232, as well as carbon dioxide 234 and hazardous air pollutants 236.

The computational operation 250 associated with the generator system 200 utilizes the following inputs 240: measured wellhead fuel supply 210, a fuel property calculator 242, and load and fuel consumption data 244. In some implementations, the measured wellhead fuel supply 210 is provided to the computational operation 250 in terms of flowrate.

At step 252, the fuel property calculator 242 relies upon a wellhead gas analysis along with the flowrate of the wellhead fuel supply 210 to determine a “Calculated Carbon Mass In”. In various implementations, the wellhead gas analysis may be supplied by the operator of the well site, or the wellhead gas analysis may be measured, either periodically or in real time.

The load and fuel consumption data 244 is a monitored engine load profile based on the type and number of generators 220 and how those generators consume fuel. That load and fuel consumption data 244 along with emission information about the internal combustion engine 254 of the generator 220 are used to determine the “Known CO and VOC Emissions” at step 256 based on the “Calculated Carbon Mass In” from step 252, where “CO” means carbon monoxide and “VOC” means volatile organic compounds.

At step 258, a difference is calculated between the “Calculated Carbon Mass In” from step 252 and the “Known CO and VOC Emissions” from step 256, resulting in the “Calculated CO2 output” at step 259, where “CO2” means carbon dioxide.

At step 260, the “Calculated CO2e output” is determined based on the “Calculated CO2 output” from step 259 and the “Known Methane Emissions” 255, which is based on how much of the VOC stream is methane. Thus, the CO2e output is based on carbon dioxide and methane, where “CO2e” means “carbon dioxide equivalent”.

Referring now to the flare stack system 300, the same measured wellhead fuel supply 210 to the generator 220 is used as the measure of wellhead fuel supply 310 to the flare stack 320. If a flare stack 320 was burning the wellhead fuel supply 310, it would produce several outputs 330, including unburnt fuel 332, carbon dioxide 334 and hazardous air pollutants 336.

The computational operation 350 associated with the flare stack system 300 utilizes the following inputs 340: the fuel supply 310 (equals the measured fuel supply 210), a fuel property calculator 342, and flare efficiency data 344. In some implementations, the fuel supply 310 is provided to the computational operation 350 in terms of flowrate.

The fuel property calculator 342 relies upon a wellhead gas analysis along with the flowrate of the wellhead fuel supply 210 to determine a “Calculated Carbon Mass In” at step 352. In various implementations, the wellhead gas analysis may be supplied by the operator of the well site, or the wellhead gas analysis may be measured, either periodically or in real time.

The flare efficiency data 344 is based on the type of flare stack 320 that is being replaced, or that would be installed, and the combustion efficiency of the specific flare stack 320. In various implementations, the flare efficiency data 344 is based at least in part on flare manufacturer data, FTIR sensor data, well site flare permit application data, operational history data, or other sources of information. That flare efficiency data 344 along with open flare combustion parameters 354 (how the flare stack 320 is operated, or would be operated, under the well site flare permit) are used to determine the “Modeled CO, CO2, NOx, VOC emissions” at step 356 based on the “Calculated Carbon Mass In” from step 352, where “CO” means carbon monoxide, “CO2” means carbon monoxide, “NOx” means nitrogen oxide, and “VOC” means volatile organic compounds.

At step 360, the “Determined CO2e Emissions” is determined based on the “Modeled CO, CO2, NOx, VOC emissions” from step 356. The CO2e emissions are based on carbon dioxide and methane.

The generator system 200 produces fewer CO2e emissions than the flare system 300. Therefore, if an operator replaces a flare system 300 with a generator system 200, or if an operator installs a generator system 200 instead of a flare system 300, the operator can generate a carbon emission reduction by removing CO2e emissions from the atmosphere or by avoiding CO2e emissions to the atmosphere. In some implementations, this carbon emission reduction generated by the operator can be used to calculate carbon credits.

The “Calculated Carbon Emission Reduction” at step 150 is the difference between the “Determined CO2e Emissions” determined at step 360 and the “Calculated CO2e output” determined at step 260. The calculated carbon emission reduction 150 corresponds to the amount of carbon dioxide equivalent reduction.

Thus, FIG. 1 illustrates an implementation of a process 100 for calculating a carbon emission reduction 150 based on a comparison between the CO2e output of a generator system 200 and the CO2e emission of a flare stack system 300 at a location, such as a subsurface well facility operated by an entity, for example.

While FIG. 1 schematically depicts both a generator system 200 and a flare stack system 300, these systems 200, 300 are not operating simultaneously. In various implementations, the generator system 200 may be a replacement for a previously installed flare stack system 300 at the premises of the entity, or the generator system 200 may be installed instead of a flare stack system 300 at the premises of the entity. In such implementations, a flow measurement device may be operable to measure, either periodically or in real time, wellhead gas as a fuel supply 210 to the generator 220 such that the computational operation 250 of the generator system 200 results in a “Calculated CO2e output” 260 based on actual data. In contrast, the measured fuel supply 210 of wellhead gas may be used as a fuel supply 310 input to the computational operation 350 of the flare stack system 300 to determine the “Determined CO2e Emissions” 360 based on the type of flare stack system 300 that was previously installed or that would have been installed.

Regardless of whether the generator system 200 and the flare stack system 300 are both operational when the computational operations 250, 350 are performed, the same quantity of wellhead gas should be used to determine the Calculated Carbon Mass In 252, 352 in both computational operations 250, 350.

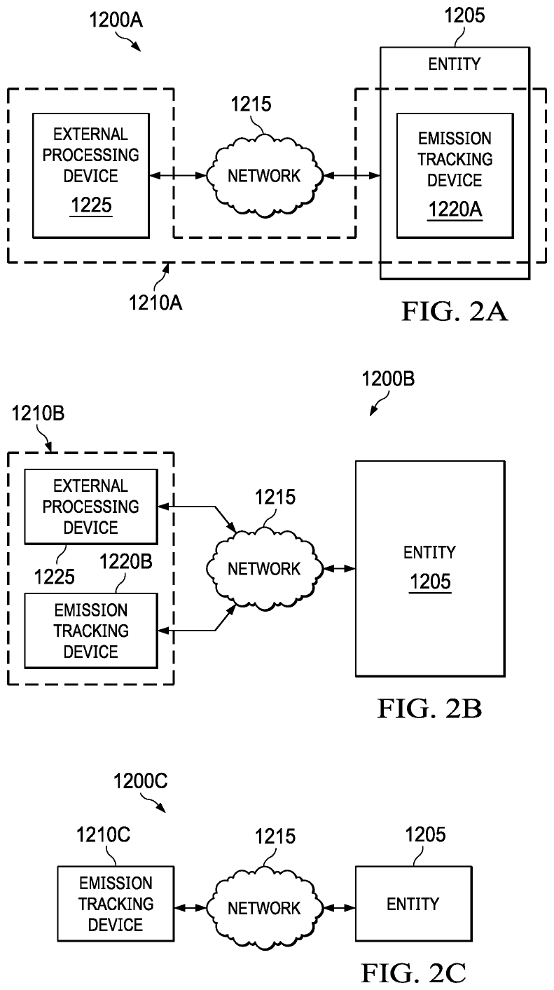

Reference is now made to FIGS. 2A-2C, which illustrate block diagrams showing various implementations of emission tracking platforms 1200A, 1200B, 1200C, respectively, that may be used to calculate the carbon emission reduction 150 according to the process 100 of FIG. 1.

As illustrated in FIG. 2A, emission tracking platform 1200A comprises an entity 1205, such as a subsurface well facility, a network 1215, and an emission tracking system 1210A. In this implementation, the emission tracking system 1210A comprises at least one emission tracking device 1220A and at least one external processing device 1225. The emission tracking device 1220A may include one or more devices, such as the volumetric flowmeter 210 described with respect to FIG. 1, that provides fuel supply data used in the computational operations 250, 350.

Network 1215 may be any network(s) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these. Network 1215 may also include a storage medium, such as, for example, a CD ROM, a DVD, an SD card, an external hard drive, a USB drive, etc.

In the illustrated implementation, the emission tracking device 1220A is located within the premises of the entity 1205, whereas the external processing device 1225 may be located separate from the emission tracking device 1220A and outside of the premises of the entity 1205. The emission tracking device 1220A is communicably coupled to the external processing device 1225 via the network 1215. The external processing device 1225 may include a data processor operable to receive data from the emission tracking device 1220A, perform calculations, such as the computational operations 250, 350 described with respect to FIG. 1, and transmit information via the network 1215 back to the entity 1205.

In the implementation illustrated in FIG. 2B, emission tracking platform 1200B also comprises an entity 1205, a network 1215, and an emission tracking system 1210B. In this implementation, the emission tracking system 1210B comprises at least one emission tracking device 1220B and at least one external processing device 1225 that are both located outside the premises of the entity 1205, while both being communicably coupled to the entity 1205 via network 1215. In this implementation, the emission tracking device 1220B is also communicably coupled to the external processing device 1225 via network 1215.

Referring now to the implementation illustrated in FIG. 2C, emission tracking platform 1200C also comprises an entity 1205, a network 1215, and an emission tracking system 1210C. In this implementation, the functionalities of an emission tracking device, such as the emission tracking devices 1220A, 1220B of FIGS. 2Aand 2B, and an external processing device, such as the external processing device 1225 of FIGS. 2A and 2B, are combined into one hardware unit indicated as emission tracking system 1210C in FIG. 2C. The emission tracking system 1210C may or may not be located within the premises of entity 1205, but the emission tracking system 1210C is communicably coupled to the entity 1205 via network 1215.

In the various implementations of FIGS. 2A-2C, the emission tracking systems 1210A, 1210B and 1210C are configured to implement the process 100 of FIG. 1 associated with the entity 1205. Tracking may include one or more non-limiting steps of monitoring, measuring, determining, modeling, analyzing, reporting and calculating the outputs, emissions, and carbon emission reduction associated with the entity 1205.

In implementations where the emission tracking devices, such as devices 1220A, 1220B of FIGS. 2A, 2B, are separate from the external processing devices, such as external processing devices 1225 of FIGS. 2A, 2B, the emission tracking devices 1220A, 1220B may be configured to carry out one or more non-limiting steps of monitoring, measuring, determining, modeling, analyzing, reporting and calculating, and the external processing device 1225 may be configured to carry out the other non-limiting steps of monitoring, measuring, determining, modeling, analyzing, reporting and calculating.

In an implementation, the emission tracking system 1210A, 1210B, 1210C comprises the device measuring the wellhead fuel supply 210 and a plurality of devices, each measuring an operational parameter of the generator 220. In an implementation, the measurements from the device measuring the wellhead fuel supply 210 and the measured operational parameters of the generator 220 are transmitted in data packages on a given time interval via telemetry to the external processing device 1225 via the network 1215. In various implementations, the given time interval may be less than 1 minute, approximately 1 minute, approximately 5 minutes, approximately 15 minutes, approximately 30 minutes, approximately 1 hour, or any other suitable time interval desired by the operator.

In an implementation, the calculated carbon emission reduction 150 is determined at each time interval. In an implementation, the calculated carbon emission reduction 150 determined at each time interval may be summed up over a designated time period to provide a measure of carbon dioxide equivalent reduction. For example, the calculated carbon emission reduction 150 determined at each time interval (such as every 15 minutes) may be summed up over a one-year time period to provide a measure of carbon dioxide equivalent reduction at the premises in tons per year.

In an implementation, the external processing device 1225 may simultaneously monitor the operation of multiple premises of the entity, wherein each of the premises has an operational generator system 200. In an implementation, the calculated carbon emission reduction 150 for one time interval at each of the premises may be summed up to provide a snapshot measurement of carbon dioxide equivalent reduction across the multiple premises. In an implementation, the summation of the calculated carbon emission reduction 150 determined at each time interval for the multiple premises may then be summed up over a designated time period to provide a measure of carbon dioxide equivalent reduction across the multiple premises for that designated time period. For example, the summation of the calculated carbon emission reduction 150 determined at each given time interval (such as every 15 minutes) may then be summed up over a one-year time period to provide a measure of carbon dioxide equivalent reduction across the multiple premises in tons per year.

It is to be understood the implementations disclosed herein are not limited to particular systems or processes described which may, of course, vary. It is also to be understood that the terminology used herein is for the purpose of describing particular implementations only, and is not intended to be limiting. As used in this specification, the singular forms “a”, “an” and “the” include plural referents unless the content clearly indicates otherwise. As another example, “coupling” includes direct and/or indirect coupling of members.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular implementations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding implementations described herein may be utilized according to the present disclosure. Accordingly, the appended claims are intended to include within their scope such processes, machines, manufacture, compositions of matter, means, methods, or steps.

What is claimed is:

1. A process for calculating a carbon emission reduction comprising:
calculating a carbon dioxide equivalent (CO2e) output associated with a quantity of wellhead gas used as a fuel supply to a generator that produces electricity;
determining a carbon dioxide equivalent (CO2e) emission associated with flaring the same quantity of wellhead gas via a flare stack system; and
calculating the carbon emission reduction based on a comparison between the calculated CO2e output and the determined CO2e emission.

2. The process according to claim 1, wherein:
calculating the carbon emission reduction comprises calculating the difference between the determined CO2e emission and the calculated CO2e output.

3. The process according to claim 1, further comprising:
measuring, in real time, the quantity of wellhead gas used as the fuel supply to the generator that produces electricity.

4. The process according to claim 3, further comprising:
using the measured quantity of wellhead gas used as the fuel supply to the generator as the same quantity of wellhead gas flared via the flare stack system.

5. The process according to claim 3, wherein:
determining the CO2e emission comprises modeling the CO2e emission using manufacturer and/or operational data associated with the flare stack system.

6. The process according to claim 1, wherein calculating the CO2e output further comprises:
calculating a first Carbon Mass based at least partially on the quantity of wellhead gas and an analysis of the wellhead gas.

7. The process according to claim 6, wherein calculating the CO2e output further comprises:
determining a Known Carbon Monoxide (CO) and Volatile Organic Compounds (VOC) Emission based at least in part on the first Carbon Mass and operational data for the generator.

8. The process according to claim 7, wherein calculating the CO2e output further comprises:
calculating a carbon dioxide (CO2) output based at least in part on the first Carbon Mass and the Known CO and VOC Emission.

9. The process according to claim 8, wherein calculating the CO2e output further comprises:
determining a Known Methane Emission based at least in part on the quantity of methane in the VOC.

10. The process according to claim 9, wherein calculating the CO2e output is based at least in part on the calculated CO2 output and the Known Methane Emission.

11. The process according to claim 1, wherein determining the CO2e emission further comprises:
determining a second Carbon Mass based at least partially on the same quantity of wellhead gas and an analysis of the wellhead gas.

12. The process according to claim 11, wherein determining the CO2e emission further comprises:
determining a Modeled carbon monoxide (CO), carbon dioxide (CO2), nitrogen oxide (NO) and Volatile Organic Compounds (VOC) Emission based at least in part on the second Carbon Mass and manufacturer and/or operational data for the flare stack system.

13. The process according to claim 12, wherein determining the CO2e emission further comprises:
determining a Modeled (CO2) Emission based at least in part on the Modeled CO, CO2, NO and VOC Emission.

14. A process for calculating a carbon emission reduction comprising:
calculating a carbon dioxide equivalent (CO2e) output associated with using a measured quantity of wellhead gas in generators to produce electricity;
determining a carbon dioxide equivalent (CO2e) emission associated with flaring via a flare stack system a quantity of wellhead gas equal to the measured quantity of wellhead gas; and
calculating the carbon emission reduction as the difference between the determined CO2e emission and the calculated CO2e output.

15. The process of claim 14, wherein:
determining the CO2e emission comprises using operational data associated with flaring via the flare stack system the quantity of wellhead gas.

16. The process of claim 14, further comprising:
periodically calculating the carbon emission reduction on a given time interval;
summing up the carbon emission reductions periodically calculated on the given time interval over a designated period of time to determine a carbon dioxide equivalent reduction over the designated period of time.
